(19) United States
(12) Patent Application Publication
(10) Pub. No.: US 2013/0204245 A1
(43) Pub. Date: Aug. 8, 2013

(54) Surface Modification of Surgical Instruments for Selective Manipulation of Biological Tissues

(71) Applicants: Albena Ivanisevic, Chapel Hill, NC (US); Tongalp H. Tezel, Louisville, KY (US)

(72) Inventors: Albena Ivanisevic, Chapel Hill, NC (US); Tongalp H. Tezel, Louisville, KY (US)

(21) Appl. No.: 13/577,427

(22) PCT Filed: Feb. 7, 2011
(86) PCT No.: PCT/US11/23926

§ 371 Date: Oct. 29, 2012

Related U.S. Application Data
(60) Provisional application No. 61/302,064, filed on Feb. 5, 2010.
(60) Provisional application No. 61/389,573, filed on Oct. 4, 2010.

(51) Int. Cl. A61B 18/00

(52) U.S. Cl. CPC A61B 18/00
USPC 606/41; 427/2.1

(57) ABSTRACT

Surgical instruments having at least one surface modified through formation of polyelectrolyte films and/or nanoparticle deposition thereon to increase adhesion between the surgical instrument and a patient's inner limiting membrane while reducing trauma during surgery are provided. The instrument includes a proximate end operable to be grasped by a surgeon and a distal tip operable to interface with a selected tissue, the distal tip comprising: i. first surface operable to receive at least one electrolyte film; ii. at least one layer of a polyelectrolyte film substantially coating the first surface, the polyelectrolyte film having at least one functional group displaying a charge.

### PRIORITY

This application claims priority to U.S. Provisional Patent Application No. 61/302,064, entitled Surface Modification of Ophthalmic Surgical Instrument for Removal of Inner Limiting Membranes, filed Feb. 5, 2010, and U.S. Provisional Patent Application No. 61/389,573, filed Oct. 4, 2010, entitled Coatings and Methods for Modifying the Surface of a Surgical Instrument, the contents of which are hereby is incorporated by reference herein in their entirety.

### BACKGROUND

Surgical manipulation of different tissues is the basis of surgical treatment of in medicine. These manipulations include grasping the tissue, incising or excising it. Surgical instruments used for these purposes are usually made from bio-inert substances such as stainless steel, titanium, and several inorganic polymers such as Teflon®, silicone, polyurethane or polyethylene. The interaction of the tips of these instruments with the biological tissues is based on mechanical squeezing or smashing action and requires a surgeon to control the interaction of the instrument with the tissue itself. Such control is an acquired skill that involves a significant learning curve, as many instrument surfaces are not optimized to adhere to the tissue that is being manipulated. On the other hand, every single tissue has its own surface characteristics and can be differentiated from the other tissues by the surface topography and composition. By way of example, the tools used to scrape the inner limiting membrane of the eye illustrates this general problem, and will be utilized herein to provide specific context to the issues and procedures herein, although it is to be understood that the concepts herein apply to a vast array of surgical instrument surfaces that are used to grasp, abrade, or pull a given tissue.

For instance, the vitreoretinal interface of the eye plays an important role in allowing the images projected and focused into a patient's eye to be received by the sensory layer of the eye—the retina. As such, abnormalities in the vitreoretinal interface can be a contributing factor in the development of several blinding conditions, such as diabetic macular edema, macular pucker and holes. One effective treatment for such disorders is to release the mechanical traction on the macula. This surgical removal of epiretinal membranes forming the macula is a procedure known as vitrectomy.

However, with current vitrectomy techniques, patches of epiretinal membrane tissue are often left postoperatively, which can lead to improper healing, and persistent issues with vision. Inner limiting membrane (“ILM”) peeling is an ancillary surgical approach that increases the success rate of epiretinal membrane (“ERM”) removal in patients suffering from diverse vitreoretinal interface abnormalities including macular hole, cystoid macular degeneration and epiretinal membranes. Indeed, several reports have shown increased closure rates of macular holes and more efficient removal of epiretinal membranes with stripping of ILM.

While ILM peeling can result in a better surgical outcome, consistent functional benefits of the procedure remain limited mainly because of the potential trauma that can be caused to underlying tissue during the procedure, and the possibility of failing to remove all of the ILM during the scraping procedure. Several intraoperative dyes used to color the tissue to be removed during ILM peeling have been proposed to allow a surgeon to better visualize the remaining ILM. These dyes reduce the operating time and the mechanical trauma to the retina by increasing the visibility of the ILM and help ensure complete removal of the ILM. However, the safety profiles of these dyes remains questionable. Enzymatic cleavage of the ILM from its underlying tissue has been another proposed a solution to these problems, but such cleavage has yet to be realized in an effective and safe procedure.

The current method of peeling the ILM involves the use of microforceps and diamond or sapphire dusted scrapers developed to peel ILM. These devices utilize the diamond or sapphire dust on the interface surface to increase the abrasive properties of the dusted surface, thereby increasing the abrasive properties of the surgical device and the adhesion between the ILM and the scraper or microforceps. However, these abrasive surfaces often fail to perforate two-micron thick ILM alone without causing any significant trauma to the underlying retina.

As such, surgical instruments modified to increase the adhesion between the ILM and the instrument to peel the ILM without creating any mechanical trauma to the underlying retina would be greatly appreciated.

### SUMMARY

The present application relates to coatings and treatments made to the tissue-engaging surfaces of surgical instruments to improve adhesion between the surgical instrument and a selected tissue. As such, this application provides for the following.

According to one embodiment, a surgical instrument addressing a selected tissue is provided, with, the instrument comprising a proximate end operable to be grasped by a surgeon and a distal tip operable to interface with a selected tissue, the distal tip comprising a first surface operable to receive at least one electrolyte film; and at least one layer of a polyelectrolyte film substantially coating the first surface, the polyelectrolyte film having at least one functional group displaying a charge.

According to at least one embodiment, the at least one layer of a polyelectrolyte film comprises alternating layers of polyelectrolytes, wherein each alternating layer of polyelectrolytes displays a charge opposite of the preceding layer of polyelectrolytes. According to an additional embodiment, the surgical instrument further comprises a first polyelectrolyte film comprising polyallylamine hydrochloride. According to at least one other embodiment, the surgical instrument further comprises a second polyelectrolyte film comprising polystyrene sulfonate.

According to at least one embodiment, a surgical instrument for addressing a selected tissue is provided, the instrument comprising a proximate end operable to be grasped by a surgeon and a distal tip operable to interface with a selected tissue, the distal tip comprising a first surface operable to receive at least one layer of nanoparticles, and at least one layer of nanoparticles adhered to the first surface, the at least one layer of nanoparticles operable to increase the adhesion force between the first surface and a target tissue. According to at least one embodiment, the at least one layer of nanoparticles comprises gold nanoparticles. Further, in at least one embodiment, the gold nanoparticles are sized between approximately 1 nM and approximately 40 nm. In another embodiment, the gold nanoparticles are sized between approximately 5 nm and approximately 20 nm. According to at least one embodiment, the gold nanoparticles are cationic gold particles. According to at least one embodiment, the gold nanoparticles are citrate-stabilized gold nanoparticles. According to at least one embodiment, the gold nanoparticles are conjugated with poly-L-lysine.

According to at least one embodiment, the surgical instrument includes at least one layer of a polyelectrolyte film substantially coating the first surface, the polyelectrolyte film having at least one functional group displaying a charge.

According to at least one embodiment, the nanoparticles are embedded within at least one of the at least one layer of a polyelectrolyte film. According to at least one embodiment, the surgical instrument is a scraper for removing an eye tissue.

According to at least one embodiment, a method of increasing the adhesion forces between a surgical instrument and a selected tissue is provided, the method comprising the step of: applying a plurality of cationic gold nanoparticles to at least one surface of the surgical instrument that contacts a patient's tissue.

According to at least one embodiment, the cationic gold nanoparticles are conjugated with one or more polyelectrolytes. According to at least one embodiment, the cationic gold nanoparticles are sized between approximately 5 nm and approximately 80 nm.

18. The method of any of claims 15-17, further comprising the step of applying at least one layer of a first polyelectrolyte film to the at least one surface of the surgical instrument that contacts a patient's eye.

19. The method of any of claims 15-18, further comprising the step of applying at least one layer of a second polyelectrolyte film to the at least one surface of the surgical instrument that contacts a patient's eye.

20. The method of claim 19, whereby the first polyelectrolyte film and the second polyelectrolyte film have opposite charges.

21. The method of claim 20, whereby the first polyelectrolyte film and the second polyelectrolyte film are applied in alternating layers.

### BRIEF DESCRIPTION OF THE DRAWINGS

FIG. 1 is a perspective view of surgical scraper.

FIG. 2 is a side diagrammatic view of a surgical instrument having a coating according to at least one embodiment herein.

FIG. 3 is a side diagrammatic view of a surgical instrument having a coating according to at least one embodiment herein.

FIG. 4 is a side diagrammatic view of a surgical instrument having a coating according to at least one embodiment herein.

FIG. 5 is a graphical representation of the test results comparing the adhesive forces between standard cantilever tips.

FIG. 6 is a graphical representation of the test results measuring the adhesive forces between surgical tips prepared utilizing a layer-by-layer approach and utilizing amine-functionalized polyelectrolytes.

FIG. 7 is a graphical representation of the test results measuring the adhesive forces between surgical tips prepared utilizing a layer-by-layer approach and utilizing hydroxyl-functionalized polyelectrolytes.

FIG. 8 is a graphical representation of the test results comparing the adhesive forces between surgical tips prepared in various methods according to at least one embodiment herein.

### DETAILED DESCRIPTION

According to the present application, a more effective way to increase adhesion between a functional surface of a surgical instrument and a selected tissue is presented. By way of nonlimiting example, the functional tip of an ocular scraper is described for illustrative purposes herein, although it will be appreciated that the embodiments and surface modifications discussed herein are applicable to a broad spectrum of surgical instruments, and include all surgical instruments that would be improved through the increased adhesion between the tissue interface surface of the instrument and a selected tissue. For instance, it will be appreciated that the methods, coatings, and structures herein may be applied to any surgical instruments utilized for manipulation of tissue in all fields of surgery, including but not limited to those surgical instruments utilized in cataract surgery, thoracic surgery, neurosurgery, urogenital surgery, orthopedic surgery, transplantation surgery, gastrointestinal surgery, skin grafting, plastic and aesthetic surgery.

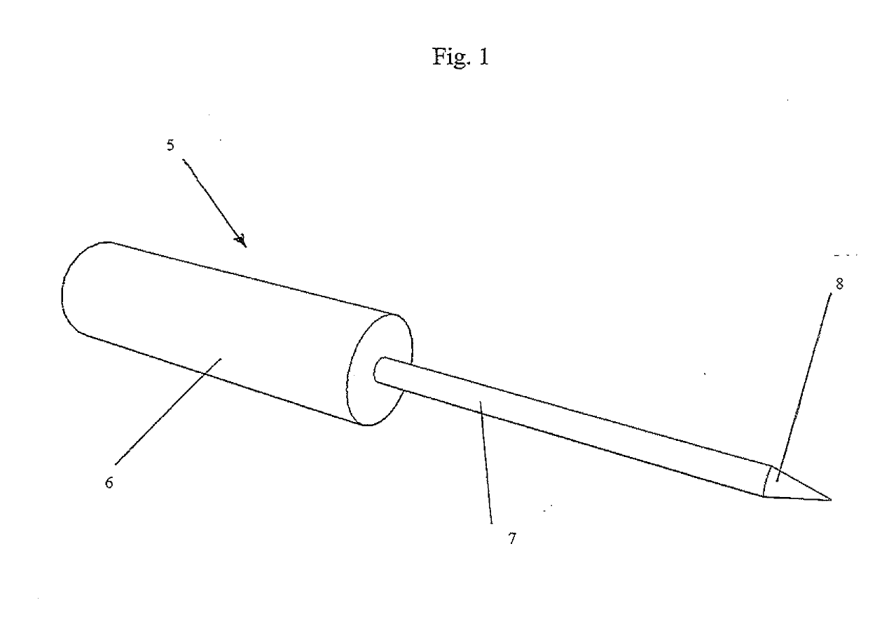

Turning now to FIG. 1, a perspective view of a surgical scraper 5 is provided according to at least one embodiment, said surgical scraper having a proximal handle 6 with a cylindrical neck 7 extending distally therefrom and terminating at its distal end with a surgical tip 8. In operation, a surgeon grasps handle 6 to manipulate surgical tip 8 such that surgical tip 8 comes in contact with a patient's tissue during surgery. For instance, a surgeon with utilize surgical scraper 5 to scrape and/or pull a patient's tissue with surgical tip 8.

According to at least one embodiment, a surgical instrument having improved surgical surface properties to increase adhesion between at least one surgical instrument surface and a patient's ILM, epi-retinal membrane (“ERM”), or other selected tissue is presented. According to at least one embodiment, at least one polyelectrolyte (“PE”) layer is adhered to a surface of a surgical instrument to increase adhesion between the surgical instrument and a patient's ILM, ERM, or other selected tissue.

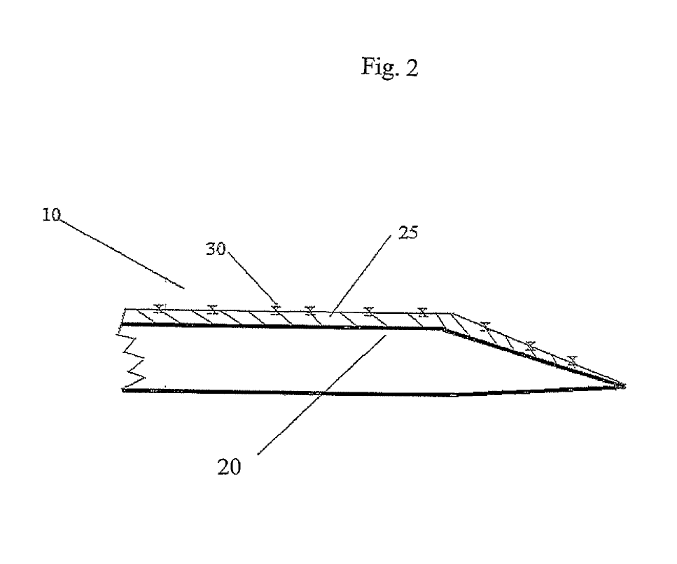

Turning to FIG. 2, a surgical instrument tip 10 for removing a selected tissue, comprises the interface between the surgical instrument such as a surgical scraper, forceps, or other instrument and a patient's tissue. According to at least one embodiment, surgical instrument tip 10, includes a surface 20 coated with at least one layer of a polyelectrolyte film 25. According to at least one embodiment, the at least one layer of polyelectrolyte film 25 may comprise any polyelectrolyte with specific functional groups such as, but not limited to, limited to, —OH, —NH2, —COOH, or a combination thereof. As shown in FIG. 1, polyelectrolyte films include functional groups 30 to increase adhesion properties between surface 20 and a patient's selected tissue.

According to at least one embodiment, a surface 20 of a surgical instrument tip 10 is coated with at least one layer of polyelectrolyte film 25 through a layer-by-layer assembly (“LbL”) or polyelectrolyte multilayer (“PEM”) film assembly. It will be appreciated LbL assembly consists in the alternate and iterative deposition of polycations and polyanions to spontaneously create multilayer thin films due to the electrostatic interactions which arise between the oppositely charged polyelectrolytes.

According to at least one embodiment, LbL films can be formed from synthetic polyelectrolytes or they may be formed from naturally occurring biological molecules, including polypeptides and polysaccharides. In at least one embodiment, LbL films can be formed from a combination of synthetic and naturally occurring polyelectrolytes. With respect to natural polyelectrolytes, LbL films grown with natural polyelectrolytes such as poly-L-lysine (PLL), hyaluronan (HA), and chitosan (CHI) may be selected if exponential growth with each deposition step is desired. Such growth may be beneficial when, for example, a surgical instrument tip has inconsistent surface features that need to be masked. With respect to synthetic polyelectrolytes, polyallylamine hydrochloride (PAH), polystyrene sulfonate (PSS), and polyacrylic acid (PAA), and other synthetic polyelectrolytes exhibiting a linear growth may be selected when the thickness and mass of the multilayer film is sought to be increased with each bilayer deposition.

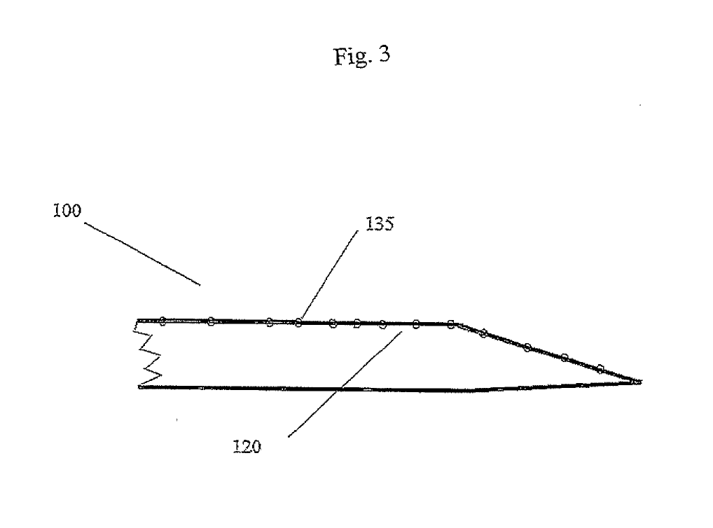

Turning now to FIG. 3, a surgical instrument 100, such as a surgical scraper, forceps, or other instrument, includes a surface 120 coated with a plurality of nanoparticles 135 are embedded into surface 120. According to at least one embodiment, nanoparticles 135 are optionally gold nanoparticles. According to at least one embodiment, nanoparticles 135 are ionically charged gold nanoparticles conjugated with synthetic and/or naturally occurring polyelectrolytes such as poly-L-lysine (PLL), hyaluronan (HA), and chitosan (CHI), polyallylamine hydrochloride (PAH), polystyrene sulfonate (PSS), and polyacrylic acid (PAA). According to certain optional embodiments, nanoparticles 135 are citrate-stabilized gold nanoparticles. According to at least one embodiment, nanoparticles 135 are sized to be about 3 nm to 80 nm in diameter, about 5 nm to 30 nm in diameter, about 10 nm to 20 nm diameter, or a combination thereof.

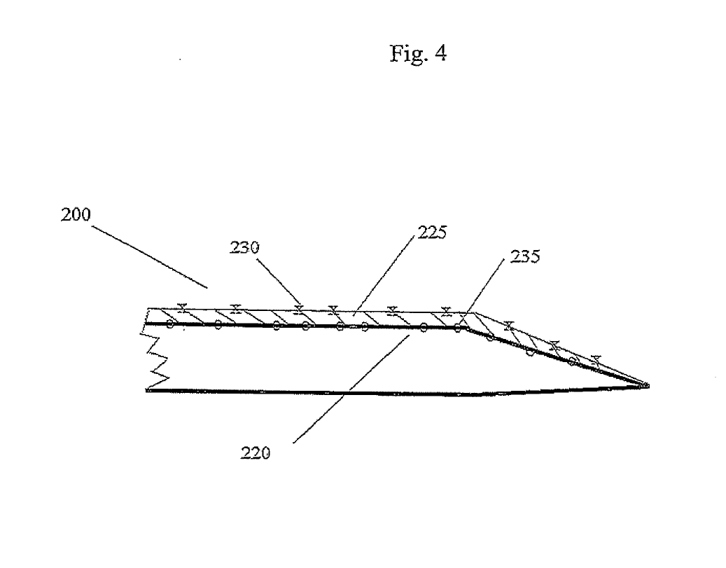

Turning now to FIG. 4, a surgical instrument 200, such as a surgical scraper, forceps, or other instrument, includes a surface 220 coated with at least one layer of a polyelectrolyte film 225, wherein a plurality of nanoparticles 235 are embedded into surface 120, and/or are embedded within the at least one layer of a polyelectrolyte film 225. According to at least one embodiment, nanoparticles 235 are optionally gold nanoparticles. According to at least one embodiment, nanoparticles 235 are ionically charged gold nanoparticles conjugated with synthetic and/or naturally occurring polyelectrolytes such as poly-L-lysine (PLL), hyaluronan (HA), and chitosan (CHI), polyallylamine hydrochloride (PAH), polystyrene sulfonate (PSS), and polyacrylic acid (PAA). According to certain embodiments, nanoparticles 235 are citrate-stabilized gold nanoparticles. According to at least one embodiment, nanoparticles 235 are optionally sized to be about 3 nm to 50 80 nm in diameter, about 5 nm to 30 nm in diameter, about 10 nm to 20 nm diameter, or a combination thereof EXEMPLARY EMBODIMENTS:

### A. Preparation and Performance of Certain Layer-By-Layer Functionalized Tips

According to at least one exemplary embodiment, multiple atomic force microscopy (“AFM”) cantilevers were prepared to test the adhesion of LBL treated surfaces to ILM. Further, according to at least one embodiment, contact-mode cantilevers were modified with amine (—NH2) and hydroxyl (—OH) groups by coating the surface using the LBL approach incorporating polystyrene sulfonate (PSS, Fischer Scientific, USA), which provided —OH groups on the surface; and polyallylamine hydrochloride (PAH, Sigma, USA), which provided —NH2 groups on the surface of the layer. In this exemplary embodiment, contact-mode tips were coated with eight polyelectrolyte layers.

Thereafter, nineteen (19) fresh surgically harvested human ILM samples from patients with stage II and stage III proliferative diabetic retinopathy were analyzed and were then utilized to test the adhesion of the abovementioned prepared tips to the harvested ILM in vitro. All ILM samples were obtained from patients during a standard-three port 25-gauge vitrectomy, and each ILM was stripped with an asymmetrical forceps (Dutch Ophthalmic USA, Exeter, N.H.) without the use of any dye. Stripped ILMs were placed on a glass slide with internal face looking up and kept at −20 C until the time of analysis.

In performing the testing, tapping-mode imaging was performed with clean silicon nitride AFM cantilevers (model: OTESPA, Veeco Probes, USA) with a nominal spring constant of 42 N/m. Liquid contact-mode imaging was performed with silicon cantilevers (MSNL-10, Veeco Proves, USA) with a nominal spring constant of 0.05 N/m. Topographical imaging of the ILM samples and treated and untreated cantilevers was performed using a MultiMode Nanoscope III equipped with Nanoscope III version 5.12r3 software. All experiments were performed under ambient conditions at scan speeds between 2 and 4 Hz. ILMs were thawed for 30 minutes before imaging, with height and deflection images obtained in air under tapping mode with silicon nitride microcantilevers (OTESPA). Five random spots were imaged on each ILM sample. At each spot, two images of different areas (1 μm2 and 25 μm2) were recorded. The roughness and surface area of the height images were measured upon a first-order flattening of the images. Surface area of the whole images was measured. Liquid contact-mode images of the ILMs were also obtained with silicon microcantilevers (MSNL-10). The samples were exposed to pure deionized (DI) water during imaging.

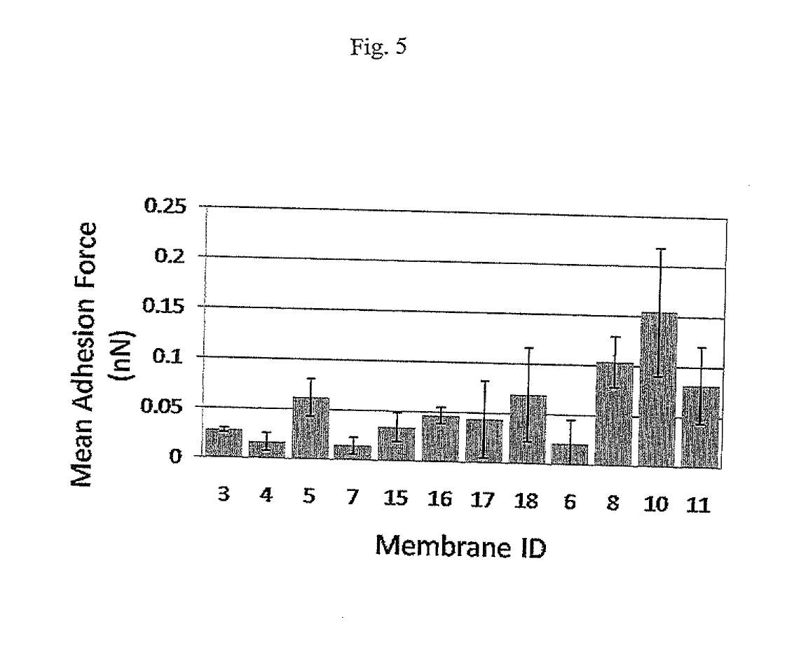

Thereafter, force measurements were obtained between clean, unmodified surfaces (such as those utilized in surgical instruments) and the ILM surfaces. These experiments were performed in force volume (FV) mode, where a force volume image is a collection of deflection versus distance curves (force curves). FV images were collected at three random spots on the ILM surface. All FV images were collected with 16 sample points per force curve and 32 force curves per line. Additionally, a topographical image of the spot was also collected at 64 sample points per line. All force measurement tests were performed in an aqueous environment, to closely resemble the physiological environment in which ILMs are removed during surgery. The FV data for the clean, unmodified surfaces are shown in FIG. 5. It will be appreciated that the FV results confirm the histological analysis of the ILM samples used in the study—that ILM tissue surfaces are highly variable both within a single sample and amongst the study samples. Specifically, a review of FIG. 5 shows a significant variability in the adhesion force between the ILM samples and the clean cantilevers, varying from approximately 0.02 nN to approximately 0.22 nN.

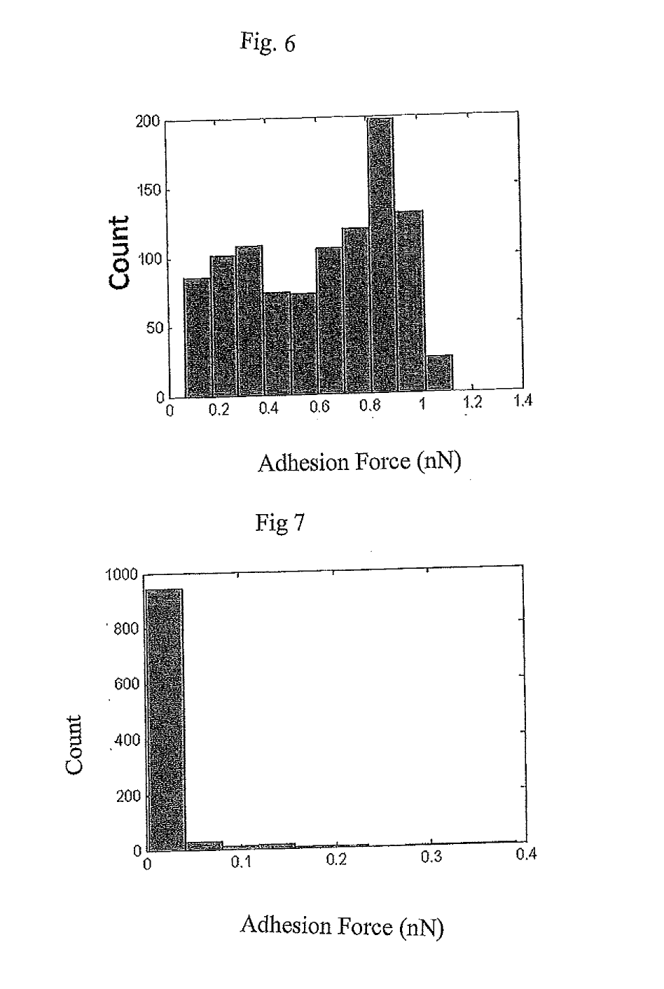

In addition, force measurements were obtained between cantilevers modified as discussed herein and the ILM surfaces. According to the present exemplary embodiment, amine-functionalized cantilevers (FIG. 6) and hydroxyl-functionalized cantilevers (FIG. 7) were tested for adhesion forces with the sample ILM. FIG. 6 displays the highly variable adhesion force, in nN between the amine-functionalized cantilevers and ILM, showing adhesion forces between approximately 0.1 nN and approximately 1.1 nN. It will be appreciated that the average adhesion force displayed between the amine-functionalized cantilever and the ILM is significantly higher than the adhesion forces between the non-functionalized cantilevers displayed in Table 1. Further, FIG. 7 displays the highly variable adhesion force, in nN, between the hodroxyl-functionalized cantilevers and ILM, showing adhesion forces between approximately 0.0 up to approximately 0.4. As such, it will be appreciated that hydroxyl-functionalized cantilevers according to at least one embodiment appear to have a repulsive effect on the surface of the ILM samples, as evidenced by the reduced adhesion forced displayed in comparison to clean cantilever tips. It should be noted that this finding is contrary to prior studies that showed that protein adsorption, film morphology, and rms roughness were independent of outer layer charge. Gong, H., et al., Interaction and adhesion properties of polyelectrolyte multilayers. Langmuir, 2005. 21(16): p. 7545-50.

B. Preparation and Testing of Certain Layer-By-Layer Functionalized Tips with Embedded Nanoparticles

According to at least one exemplary embodiment, multiple atomic force microscopy (“AFM”) cantilevers were prepared to test the adhesion of LBL and nanoparticle treated surfaces to ILM. First, tipless AFM cantilevers were silanized to facilitate the absorption of unconjugated gold nanoparticles (“AuNPs”) with negative surface charge onto the cantilever surface. AFM probes were immersed in subsequent baths of deionized water, ethanol, and methanol to clean the surface. Subsequently, the cantilevers were placed in piranha solution (3:1 v/v Sulfuric Acid:Hydrogen Peroxide 30%) for 10 minutes to remove impurities and increase hydrophilicity of the surface. Probes were rinsed in ethanol and dried with nitrogen. Next, the probes were placed in 2 mL of APTES on a Pyrex® glass petri dish and placed in a reaction chamber in a nitrogen gas environment for two to three hours. This allowed for the deposition of the silane groups on the cantilever surface. The probes were removed from the chamber and washed in ethanol and dried once again. Finally, the cantilevers were dried in an oven set at 120° C. for 20 minutes, ensuring the dehydration of the silane film on the surface. The probes were washed and stored in a dry environment until further use.

Further, according to at least one embodiment, a 4‰ solution of unconjugated AuNPs of two different diameters (5 and 20 nm, obtained from Ted Pella, Inc.) was prepared by washing AuNPs by centrifugation. The supernatant was removed and 1 mL of deionized water was added. The silanized probes were immersed in this solution for 10 minutes prior to experimentation.

Further, according to at least one embodiment, two polyelectrolytes were utilized to prepare a polyelectrolyte film on the abovementioned surface: polystyrene sulfonate (PSS, Polysciences, USA), which provided (—OH) groups to the surface and polyallylamine hydrochloride (PAH, Sigma, USA), which provided the (—NH2) groups for precationic poly-L-lysine, (“PLL”) and unconjugated gold nanoparticles. In particular, AFM cantilever surfaces were pre-treated by immersion in piranha solution. Solutions of PAH and PSS with concentrations of 1 mg/mL were prepared by dissolving the polyelectrolytes in deionized water. All cantilevers were first dipped in the PAH solution as the surfaces were made hydrophilic by the bathing in piranha solution. LbL films were created by sequentially immersing the cantilevers in solutions of PAH and PSS. Each immersion lasted 10 minutes. After each dipping, the surfaces were washed in deionized water and dried. Two LbL configurations were created: (PAH/PSS)₄ and (PAH/PSS)₄/PAH, with negatively charged and positively charged outer layers, respectively.

Cationic AuNPs were embedded in the LbL films to impart these with more variable topographies. Four parts per mille solutions (4‰) of AuNPs were prepared by mixing centrifuged and washed AuNPs with 1 mL of PSS solution. The final AuNP/PSS solution was used to form the last PSS layer of the films.

Thereafter, nineteen (19) fresh surgically harvested human ILM samples from patients with stage II and stage III proliferative diabetic retinopathy were analyzed and were then utilized to test the adhesion of the abovementioned prepared tips to the harvested ILM in vitro. All ILM samples were obtained from patients during a standard-three port 25-gauge vitrectomy, and each ILM was stripped with an asymmetrical forceps (Dutch Ophthalmic USA, Exeter, N.H.) without the use of any dye. Stripped ILMs were placed on a glass slide with internal face looking up and kept at −20 C until the time of analysis.

In performing the testing, tapping-mode imaging was performed with clean silicon nitride AFM cantilevers (model: OTESPA, Veeco Probes, USA) with a nominal spring constant of 42 N/m. Liquid contact-mode imaging was performed with silicon cantilevers (MSNL-10, Veeco Proves, USA) with a nominal spring constant of 0.05 N/m. Topographical imaging of the ILM samples and treated and untreated cantilevers was performed using a MultiMode Nanoscope III equipped with Nanoscope III version 5.12r3 software. All experiments were performed under ambient conditions at scan speeds between 2 and 4 Hz. ILMs were thawed for 30 minutes before imaging, with height and deflection images obtained in air under tapping mode with silicon nitride microcantilevers (OTESPA). Five random spots were imaged on each ILM sample. At each spot, two images of different areas (1 μm2 and 25 μm2) were recorded. The roughness and surface area of the height images were measured upon a first-order flattening of the images. Surface area of the whole images was measured. Liquid contact-mode images of the ILMs were also obtained with silicon microcantilevers (MSNL-10). The samples were exposed to pure deionized (DI) water during imaging.

These cantilevers have a 10 nm radius with a height between 2.5 and 8 nm. The cantilever tip can be seen as a microscale topographical feature. The addition of 5 and 20 nm AuNPs could decrease the contact area of the tip to the ILM, thus, decreasing the adhesion measured. A tipless cantilever was therefore chosen for these experiments. These types of cantilevers have the advantage of being customizable. The flat surface assures that any modification made will come in contact with the sample to be imaged. For the force volume experiments, this translates into a higher contact area between the LbL-AuNP modification and the ILM sample.

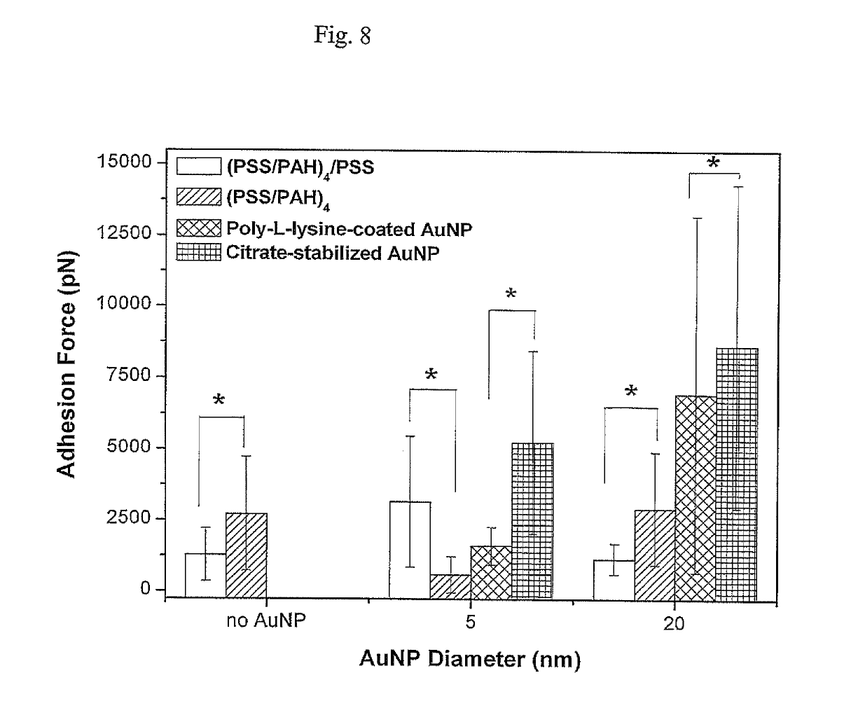

Table 1 details the topographical description of the modification configurations tested, while FIG. 8 details the mean adhesion forces for each of the modification configurations tested. (PAH/PSS)4 and (PAH/PSS)4/PAH films alone, as well as tipless cantilevers modified with cationic AuNPs (conjugated with poly-L-lysine) and citrate-stabilized AuNPs were chosen as controls to which compare the LbL-AuNP assemblies.

Films terminated with PAH showed a higher adhesion to ILM samples than PSS-terminated films. This is consistent with results described in Example 1 above. With the addition of 5 nm cationic AuNPs to the films, the aforementioned behavior is reversed. Indeed, PSS-terminated films with 5 nm AuNPs show higher adhesion than the PAH-terminated films with the same particles. In the case of 5 nm AuNPs only (particles adsorbed on the cantilever surface), citrate-stabilized AuNPs showed higher binding forces to the ILMs than poly-L-lysine coated particles of the same diameter. Negatively charged, citrate-stabilized AuNPs (5 nm) showed higher affinity for ILM surfaces than the LbL-AuNP (5 nm) modified cantilevers. With regards to modifications which included 20 nm AuNPs, PAH-terminated films showed higher adhesion forces than its PSS-terminated counterparts. Once again, cantilever surfaces modified only with AuNPs showed greater adhesion forces than surfaces modified with LbL-AuNP assemblies. Indeed, these AuNP modified tips showed multiple bonding events, indicative of the modification having a significant effect on adhesion between the ILM and the cantilever. It also illustrates that the binding events are not random adhesions.

TABLE 1
Topographical data of ILMs utilized
for adhesion studies with AFM

| | Image Area (μm²) | | | |
|---|---|---|---|---|
| | 1 × 1 | | 5 × 5 | |
| | RMS | Surface | RMS | Surface |
| Membrane | Roughness | Area | Roughness | Area |
| ID | (nm) | (□m²) | (nm) | (□m²) |
| 1 | 25.4 ± 6.5 | 1.1 ± 0.1 | 102.2 ± 36.1 | 30.0 ± 2.2 |
| 2 | 23.7 ± 10.6 | 1.1 ± 0.02 | 65.5 ± 24.9 | 25.9 ± 0.7 |
| 4 | 41.1 ± 36.0 | 0.5 ± 0.05 | 130.0 ± 40.0 | 12.4 ± 0.9 |
| 6 | 12.0 ± 5.0 | 0.5 ± 0.05 | 44.0 ± 10.0 | 12.1 ± 0.02 |
| 8 | 6.3 ± 2.1 | 0.4 ± 0.1 | 26.4 ± 8.3 | 11.8 ± 0.3 |
| 11 | 16.1 ± 17.0 | 0.5 ± 0.1 | 55.2 ± 28.0 | 12.0 ± 1.0 |

n ≧ 5 for each value

According to the above embodiments, cantilever surfaces modified only with AuNPs showed higher adhesion forces than LbL or LbL-AuNP modified cantilevers. Citrate-stabilized AuNPs with a net negative charge had the highest affinity for ILM surfaces at both 5 nm and 20 nm. In each of these cases, higher diameter AuNPs seemed to increase adhesion forces, independent of outer layer charge. As a natural peptide with a positive charge, PLL could be expected to increase adhesion to a cell surface, as has been noted in multiple studies (REF). LbL films containing PLL as a building block are more effective in increasing adhesion to cells when crosslinked. On its own and as a coating for a hard spherical particle, PLL could be capable of increasing the adhesion force due to roughness and charge more than embedded in a LbL film.

No significant differences in adhesion forces were found between the PAH-terminated films without particles and PAH-terminated films with particles. The particle charge in this case is not a factor, as the particles were mixed into the PSS solution prior to depositing them onto the assembly. Even though the LbL-AuNP films displayed a higher roughness than just the adsorbed particles, the embedding of the particles into the film may cause a loss of roughness by “softening” the effect of the asperities represented by the nanoparticles. Imaging of the AuNP-modified cantilevers showed that particles were found in clumps on the surface and their distribution was not homogeneous throughout the surface.

Given the above examples, it will be appreciated that surgical instruments such as forceps and scrapers, and particularly surgical instruments utilized to scrape and pull tissues tissue would benefit from the addition of polyelectrolyte films, gold nanoparticles, or both being added to at least one surface of those surgical instruments that comes in contact with a patient's tissue.

Further provided, in some embodiments of the presently-disclosed subject matter, are surgical instruments for peeling or scraping membranes or tissues. In some embodiments, a surgical instrument for peeling or scraping membranes or tissues is provided that comprises a plurality of nanoparticles and a multilayer polyelectrolyte film adhered to a surface of the surgical instrument. Again, in these embodiments, the nanoparticles, the multilayer polyelectrolyte film, or both have a charge and a nanoscale roughness to thereby increase the adhesion and affinity of the surface of the surgical instrument to the inner limiting membrane.

It will be understood that various details of the presently disclosed subject matter can be changed without departing from the scope of the subject matter disclosed herein. Furthermore, the foregoing description is for the purpose of illustration only, and not for the purpose of limitation.

1. A surgical instrument addressing a selected tissue, the instrument comprising:
a. a proximate end operable to be grasped by a surgeon and a distal tip operable to interface with a selected tissue, the distal tip comprising:
i. first surface operable to receive at least one electrolyte film;
ii. at least one layer of a polyelectrolyte film substantially coating the first surface, the polyelectrolyte film having at least one functional group displaying a charge.

2. The surgical instrument of claim 1, wherein the at least one layer of a polyelectrolyte film comprises alternating layers of polyelectrolytes, wherein each alternating layer of polyelectrolytes displays a charge opposite of the preceding layer of polyelectrolytes.

3. The surgical instrument of claim 1, further comprising a first polyelectrolyte film comprising polyallylamine hydrochloride.

4. The surgical instrument of claim 3, further comprising a second polyelectrolyte film comprising polystyrene sulfonate.

5. A surgical instrument for addressing a selected tissue, the instrument comprising:
a. a proximate end operable to be grasped by a surgeon and a distal tip operable to interface with a selected tissue, the distal tip comprising:
i. a first surface operable to receive at least one layer of nanoparticles;
ii. at least one layer of nanoparticles adhered to the first surface, the at least one layer of nanoparticles operable to increase the adhesion force between the first surface and a target tissue.

6. The surgical instrument of claim 5, wherein the at least one layer of nanoparticles comprises gold nanoparticles.

7. The surgical instrument of claim 6, wherein the gold nanoparticles are sized between approximately 1 nm and approximately 40 nm.

8. The surgical instrument of claim 6, wherein the gold nanoparticles are sized between approximately 5 nm and approximately 20 nm.

9. The surgical instrument of claim 6, wherein the gold nanoparticles are cationic gold particles.

10. The surgical instrument of claim 6, wherein the gold nanoparticles are citrate-stabilized gold nanoparticles.

11. The surgical instrument of claim 6, wherein the gold nanoparticles are conjugated with poly-L-lysine.

12. The surgical instrument of claim 6, further comprising at least one layer of a polyelectrolyte film substantially coating the first surface, the polyelectrolyte film having at least one functional group displaying a charge.

13. The surgical instrument of claim 6, wherein the nanoparticles are embedded within at least one of the at least one layer of a polyelectrolyte film.

14. The surgical instrument of claim 6, wherein instrument is a scraper for removing an eye tissue.

15. A method of increasing the adhesion forces between a surgical instrument and a selected tissue, the method comprising the step of:
a. applying a plurality of cationic gold nanoparticles to at least one surface of the surgical instrument that contacts a patient's tissue.

16. The method of claim 15, wherein the cationic gold nanoparticles are conjugated with one or more polyelectrolytes.

17. The method of claim 15, wherein the cationic gold nanoparticles are sized between approximately 5 nm and approximately 20 nm.

18. The method of claim 15, further comprising the step of applying at least one layer of a first polyelectrolyte film to the at least one surface of the surgical instrument that contacts a patient's eye.

19. The method of claim 15; further comprising the step of applying at least one layer of a second polyelectrolyte film to the at least one surface of the surgical instrument that contacts a patient's eye.

20. The method of claim 19, whereby the first polyelectrolyte film and the second polyelectrolyte film have opposite charges.

21. The method of claim 20, whereby the first polyelectrolyte film and the second polyelectrolyte film are applied in alternating layers.
